SEQUENCE LISTING (2 sequences)

SEQ ID NO: 1 - protein, 13 aa, Homo sapiens
SYSMEHFRWGKPV

SEQ ID NO: 2 - protein, 13 aa, Artificial
  alpha-MSH analogue
SYSXEHXRWGKPV

(19) United States
(12) Patent Application Publication
(10) Pub. No.: US 2014/0050773 A1
(43) Pub. Date: Feb. 20, 2014

(54) COMPOUND COMPRISING ALPHA-MSH FOR USE IN ENDODONTIC REGENERATION

(71) Applicants: Nadia Benkirane-Jessel, Strasbourg Cedex (FR); Carlos Mendoza Palomares, Bordeaux (FR); Florence Fioretti, Strasbourg (FR)

(72) Inventors: Nadia Benkirane-Jessel, Strasbourg Cedex (FR); Carlos Mendoza Palomares, Bordeaux (FR); Florence Fioretti, Strasbourg (FR)

(73) Assignee: Universite De Strasbourg, Strasbourg (FR)

(21) Appl. No.: 13/990,004

(22) PCT Filed: Nov. 26, 2010
(86) PCT No.: PCT/IB10/03458

§ 371 Date: Oct. 16, 2013

(51) Int. Cl. A61K 38/22; A61K 9/70

(52) U.S. Cl. CPC A61K 38/22; A61K 9/7007
USPC 424/443; 514/10.7

(57) ABSTRACT

The present invention concerns a compound comprising an α-MSH peptide, coupled to a polypeptide consisting of a chain of about 15 to about 400 amino acids, for use in endodontic regeneration and/or for the treatment of dental inflammatory diseases. The invention further concerns pharmaceutical compositions, in particular nanostructured compositions, comprising such a compound.

### CROSS-REFERENCE TO RELATED APPLICATIONS

This application is a United States National Stage application of International Patent Application No. PCT/IB2010/003458, filed on Nov. 26, 2010, which is incorporated by reference herein in its entirety.

### INCORPORATION OF SEQUENCE LISTING

A text file of the Sequence Listing contained in the file named “114294_Sequence.txt” which is 872 bytes (measured in MS-Windows®) in size and which was created on May 28, 2013, is electronically filed herewith and is incorporated by reference in its entirety. This Sequence Listing consists of SEQ ID NO:1-2.

### BACKGROUND OF THE INVENTION

The present invention concerns a compound comprising an α-MSH peptide, coupled to a polypeptide consisting of a chain of about 15 to about 400 identical amino acids, for use in endodontic regeneration and/or for the treatment of dental inflammatory diseases. The invention further concerns pharmaceutical compositions, in particular nanostructured compositions, comprising such a compound.

Alpha-Melanocyte Stimulating Hormone (α-MSH) Peptides

Alpha-melanocyte stimulating, hormone peptides (also known as α-MSH, or melanocortin peptides) are crucial in cutaneous biology, but also involved in other tissues metabolism as adipose tissue and bone. α-MSH have been shown to possess anti-inflammatory effects in many experimental models of acute and chronic inflammation (Catania et al., Trends Endocrinol. Meta. 2000, 11:304-308; Catania et al., Pharmacol. Rev., 2004, 56:1-29; Lam and Getting Curr. Drug. Targets Inflamm. Allergy, 2004, 3:311-315), a MSH is also able to reduce tissue fibrosis. To date, five melanocortin receptors (MCR) coupled to adenylate cyclase have been identified based on their ability to increase intracellular cAMP when activated. α-MSH have been shown to inhibit the production and activity of pro-inflammatory mediators such as IL-1, TNF-α, IL-6, IL-13, and KC, the mouse homolog of human chemokine gro-α as well as the expression of adhesion molecules such as ICAM-1. They also induce the production of the anti-inflammatory cytokine, IL-10. These effects occur primarily by inhibiting IκBα degradation and NF-κB activation.

The above effects have been characterized in monocytes, keratinocytes and dermal fibroblasts. In particular, differents studies reported the anti-inflammatory effects of α-MSH on dermal fibroblasts and shown not only the inhibition of TNF-α signaling but also the suppression of the TGF-β1-induced expression of collagen. This combined anti-inflammatory and anti-fibrogenic activity of α-MSH on this human fibroblastic cell type is of special value for the therapy of skin disorders where fibroblasts are aberrantly activated (Hill et al. Peptides. 2005; 26:1150-1158; Hedley et al. Cell. Res. 2002, 15:49-56; Schiller et al., J. Biol. Chem. 2009, 26:1-22).

Previous studies have also shown that α-MSH can be covalently coupled to poly(l-lysine) and incorporated into multilayer films, without changes in their biological properties (Chluba et al. Biomacromolecules. 2001, 2:800-805), and that such α-MSH derivatives retain anti-inflammatory properties in vitro (Jessel at al. Adv. Materials, 2004, 16:1507-1511; Jessel at al. Adv. Functional Materials, 2004, 14:174-182). In Schultz at al. (Biomaterials. 2005, 26:2621-2630), the α-MSH derivatives were coupled to the carrier polyion poly-l-glutamic acid (PGA) which leaves the anti-inflammatory C-terminal sequence Lys11-Pro12-Val13 of α-MSH peptides accessible. This PGA-α-MSH multilayer film was shown to have anti-inflammatory effects in vivo in a model of tracheal prosthesis.

Current Treatments of Dental Inflammatory Diseases

In teeth with periradicular lesions, the removal of the irritants (inflamed or necrotic tissue) from the root canal system begins the process of repair and resolution. Repair of periradicular lesions is primarily characterized by inflammatory cell infiltration which is responsible for removal endogenous and exogenous irritants, followed by fibroblastic proliferation collagen deposition, bone formation and cement apposition when root desorption occurs.

Most periapical diseases are induced as a result of direct or indirect involvement of oral bacteria. In most cases the aetiological factors are oral contaminants through the root canal or degenerating pulpal tissues. Because bacteria initiate and perpetuate the pulpal tissues, it is needed to control not only bacterial progression in pulpal and periapical diseases but also the responses of pulpal and periapical tissues to bacterial infection. The control of the pulp fibroblast response is fundamental to control the pulp inflammation.

More generally, the vitality of the pulp is fundamental to the functional life of the tooth and is a priority for targeting clinical management strategies. Dental pulp, as any other connective tissue, responds to aggression with the inflammation process, in order to eliminate pathogens and allow repair. However, due to its particular features as the confinement in hard chamber and its unique blood irrigation and lymphatic circulation, a pulp inflammation process becomes hard to control and dissipate. Frequently the pulp inflammation is so painful and clinically irreversible that the removal of the whole pulp is required.

Pulp fibroblasts are a key group of dental cells responsible for controlling a variety of matritial processes following dental injury. They play a central role in signalling various aspects of tissue regeneration. The control of the pulp fibroblast response is fundamental to control the pulp inflammation (Witherspoon Pediatr. Dent, 2008, 30:220-224; Goldberg et al., Pharmacol. Res. 2008, 58:137-147; Wisithphrom and Windsor J. Endod. 2006, 32:853-861).

Millions of teeth are saved each year by root canal therapy. However, such a treatment involves pulp devitalization. An ideal form of therapy would consist of a regenerative approach in which diseased or necrotic pulp tissues are replaced with healthy pulp tissue to revitalize teeth. There is thus a need in the art, both in human medicine and in veterinary medicine, for a dental treatment allowing endodontic regeneration.

### DESCRIPTION OF THE INVENTION

The inventors have studied the effects of PGA-α-MSH on dental pulp fibroblasts. Lipopolysaccharide (LPS)-stimulated fibroblasts incubated with PGA-α-MSH showed an early time-dependent inhibition of TNF-α, a late induction of IL-10 and no effect on IL-8 secretion. However, in the absence of LPS, PGA-α-MSH induced IL-8 secretion and proliferation of pulp fibroblasts, whereas free α-MSH inhibited this proliferation. Thus. PGA α-MSH promotes human pulp fibroblast adhesion and cell proliferation. It can also reduce the inflammatory state of LPS-stimulated pulp fibroblasts observed in gram negative bacterial infections. To understand more these results, multilayered polyelectrolytes films have been used as a reservoir for PGA-α-MSH by using not only PLL (poly L-Lysine), but also the Dendri Graft Poly-L-Lysines (DGL^{G4}) to be able to adsorb more PGA-α-MSH. It was found that by using PGA-α-MSH, not only the viability of cells but also the proliferation was increased. These nanostructured architecture were analyzed at the nanoscale by Atomic Force microscopy and an increase of thickness and roughness was observed in the presence of PGA-α-MSH incorporated into the multilayered film (PLL-PGA-α-MSH)₁₀ or (DGL^{G4}-PGA-α-MSH)₁₀ in accordance of the increase of the proliferation of the cells growing on the surface of these architectures.

More specifically, the inventors have surprisingly found that while the presence of α-MSH alone or of a poly(glutamic acid) polypeptide (PGA) alone inhibits proliferation of pulp fibroblasts, the presence of α-MSH coupled to PGA (α-MSH-PGA) promotes proliferation of pulp fibroblasts (see FIG. 4). In addition, it has unexpectedly been found that α-MSH-PGA promotes adhesion of pulp fibroblasts more efficiently than α-MSH alone or PGA alone (see FIGS. 5, 6 and 7).

Since α-MSH-PGA promotes proliferation and adhesion of pulp fibroblasts in addition to exhibiting anti-inflammatory effects, it can advantageously be used for endodontic regeneration, e.g. in the frame of pulp capping. In particular, it can be used for regenerating the pulp of teeth, and thus avoid pulp devitalization, treatment by root canal therapy and/or tooth extraction.

Compounds for Use According to the Invention

Therefore, the invention pertains to compounds comprising an alpha-melanocyte stimulating hormone (α-MSH) peptide, coupled to a polypeptide consisting of a chain of about 15 to about 400 identical amino acids (further referred to as “compounds according to the invention”). According to the invention, these compounds are used in endodontic regeneration (in particular for regenerating the pulp of teeth).

The compounds according to the invention are capable of inducing and/or increasing adhesion and/or proliferation of human pulp fibroblasts. Determining whether a compound is capable of inducing and/or increasing adhesion and/or proliferation of human pulp fibroblasts can for example be assessed using the protocols described in Examples 1 and 5. More specifically, pulp fibroblast proliferation can for example be assessed using an acid phosphatase assay. Pulp fibroblast adhesion can for example be assessed through confocal microscopy, which allows analyzing cell morphology and/or quantifying adherent cells.

The compounds according to the invention comprise an α-MSH peptide. As used herein, the terms “alpha-melanocyte stimulating hormone peptide” and ‘α-MSH peptide” encompass biologically active naturally-occurring alpha-melanocyte stimulating hormone peptides as well as biologically active variants and analogues thereof.

“Naturally-occurring” α-MSH peptides can be of any origin. Preferably, the α-MSH peptide is of human origin. A sequence of a human α-MSH peptide is shown as SEQ ID NO: 1 (Ser-Tyr-Ser-Met-Glu-His-Phe-Arg-Trp-Gly-Lys-Pro-Val).

“Variants” of naturally-occurring α-MSH peptides correspond to peptides that comprise at least one mutation compared to a naturally-occurring α-MSH peptide, and/or to peptides comprising or consisting of a fragment of a naturally-occurring or mutated α-MSH peptide.

By “analogue” (also referred to as peptidomimetic) is meant a variant that comprises at least one modified and/or unusual amino acid, and/or at least one chemical modification. Such analogues are known in the art and include, e.g., those described in Haskell-Luevano et al. (J Med Chem. 1997 40:2133-9).

As used herein, the term “peptide” refers to a polypeptide that has a length of at most 50 amino acids (e.g. of about 3 to about 40, of about 3 to about 20, of about 8 to about 40, of about 8 to about 30 or of about 8 to about 20, of about 10 to about 15 amino acids). By “polypeptide” is meant a polymer of amino acids, preferably linked by peptide bonds. The amino acids of a polypeptide may correspond to naturally-occurring amino acids (encoded by the genetic code as well as unusual amino acids) and/or to modified amino acids.

In a preferred embodiment, the compound according to the invention comprises an α-MSH peptide that comprises or consists of:
a) a sequence of SEQ ID NO: 1 or SEQ ID NO: 2; and/or
b) a sequence homologous to SEQ ID NO: 1 or SEQ ID NO: 2, wherein said homologous sequence is at least 60%, 65%, 70%, 75%, 80%, 85%, 90% or 95% identical to a sequence of SEQ ID NO: 1 or SEQ ID NO: 2. That homologous sequence preferably comprises amino acids 11 to 13 of SEQ ID NO: 1 or SEQ ID NO: 2. Indeed, these three amino acids located at the C-terminal extremity of α-MSH peptides are known to be responsible of the anti-inflammatory activity of α-MSH peptides; and/or
c) a fragment of at least 8 consecutive amino acids of the sequence of (a) or (b), wherein said fragment comprises amino acids 11 to 13 of SEQ ID NO: 1 or SEQ ID NO: 2.; and/or
d) a fragment of at least 8 consecutive amino acids of the sequence of (a) or (b), wherein said fragment comprises amino acids 6 to 13 of SEQ ID NO: 1, SEQ ID NO: 2 or of the sequence homologous thereto.

In a most preferred embodiment according to the invention, the compound according to the invention comprises or consists of an α-MSH peptide comprising or consisting of a sequence of SEQ ID NO: 2 (i.e. Ser-Tyr-Ser-Nle-Glu-His-_{D}-Phe-Arg-Trp-Gly-Lys-Pro-Val).

The α-MSH peptides in accordance with the invention are biologically active. By a “biologically active” α-MSH peptide is meant a peptide that exhibits anti-inflammatory activity. Determining whether a compound exhibits anti-inflammatory activity can for example be assessed using the protocols described in Examples 1 and 2. More specifically, the anti-inflammatory activity can for example be assessed through monitoring the levels of an anti-inflammatory cytokines such as IL-10.

By a peptide having an amino acid sequence at least, for example, 95% “identical” to a query amino acid sequence of the present invention, it is intended that the amino acid sequence of the subject peptide is identical to the query sequence except that the subject peptide sequence may include up to five amino acid alterations per each 100 amino acids of the query amino acid sequence. In other words, to obtain a peptide having an amino acid sequence at least 95% identical to a query amino acid sequence, up to 5% (5 of 100) of the amino acid residues in the subject sequence may be inserted, deleted, or substituted with another amino acid.

In the frame of the present application, the percentage of identity is calculated using a global alignment (i.e. the two sequences are compared over their entire length). Methods for comparing the identity of two or more sequences are well known in the art. The <<needle>> program, which uses the Needleman-Wunsch global alignment algorithm (Needleman and Wunsch, 1970 J. Mol. Biol. 48:443-453) to find the optimum alignment (including gaps) of two sequences when considering their entire length, may for example be used. The needle program is for example available on the ebi.ac.uk world wide web site. The percentage of identity in accordance with the invention is preferably calculated using the EMBOSS::needle (global) program with a “Gap Open” parameter equal to 10.0, a “Gap Extend” parameter equal to 0.5, and a Blosum62 matrix.

Peptides consisting of an amino acid sequence “at least 60%, 70%, 80%, 85%, 90%, 95% or 99% identical” to a reference sequence may comprise mutations such as deletions, insertions and/or substitutions, preferably substitutions, compared to the reference sequence.

in case of substitutions, the peptide consisting of an amino acid sequence at least 60%, 65%, 70%, 75%, 80%, 85%, 90% or 95% identical to a reference sequence may correspond to a homologous sequence derived from another species than the reference sequence, or to a peptide coded by another allele of the corresponding gene.

In a preferred embodiment, at least one or all substitutions correspond to a conservative substitution as indicated in the table below,

| Conservative substitutions | Type of Amino Acid |
|---|---|
| Ala, Val, Leu, Ile, Met, Pro, Phe, | Amino acids with aliphatic |
| Trp | hydrophobic side chains |
| Ser, Tyr, Asn, Gln, Cys | Amino acids with uncharged but |
| | polar side chains |
| Asp, Glu | Amino acids with acidic side chains |
| Lys, Arg, His | Amino acids with basic side chains |
| Gly | Neutral side chain |

In another preferred embodiment, at least one or all substitutions correspond to a replacement of an amino acid with a modified and/or unusual amino acid, e.g. a replacement of an amino acid with an unusual amino acid like Nle, Nva or Orn.

In still another preferred embodiment, at least one or all substitutions correspond to a replacement of an amino acid with an amino acid having a different chirality. A naturally occurring amino acid (that is in form) may for example be replaced with the corresponding d form (D-enantiomer), or with the corresponding d form together with an inversion of the amino acid chain (from the C-terminal end to the N-terminal end).

The peptides according to the invention may further comprise chemical modifications such as modifications improving their stability and/or their biodisponibility. Such chemical modifications aim at obtaining peptides with increased protection of the peptides against enzymatic degradation in vivo, and/or increased capacity to cross membrane barriers, thus increasing its half-life and maintaining or improving its biological activity. Any chemical modification known in the art can be employed according to the present invention. Such chemical modifications include but are not limited to:
modifications to the N-terminal and/or C-terminal ends of the peptides such as e.g. N-terminal acylation (preferably acetylation) or desamination, or modification of the C-terminal carboxyl group into an amide or an alcohol group;
modifications at the amide bond between two amino acids: acylation (preferably acetylation) or alkylation (preferably methylation) at the nitrogen atom or the alpha carbon of the amide bond linking two amino acids;
modifications at the alpha carbon of the amide bond linking two amino acids such as e.g. acylation (preferably acetylation) or alkylation (preferably methylation) at the alpha carbon of the amide bond linking two amino acids;
azapeptides, in which one or more alpha carbons are replaced with nitrogen atoms; and
betapeptides, in which the amino group of one or more amino acid is bonded to the β carbon rather than the α carbon.

In addition to the α-MSH peptide, the compound according to the invention comprises a polypeptide consisting of a chain of about 15 to about 400 amino acids.

The polypeptide according to the invention may for example comprise about 25 to about 300 amino acids, about 50 to about 200 amino acids, about 75 to about 150 amino acids, about 90 to about 120 amino acids, or about 100 or 110 amino acids.

The polypeptide according to the invention can be comprised of any type of amino acids. However, in a specific embodiment, the polypeptide is comprised of amino acid having charged side chains, such as aspartic acids (negatively charged), glutamic acids (negatively charged), lysines (positively charged), arginines (positively charged) and histidines (positively charged).

In a preferred embodiment, the polypeptide according to the invention is a polyanion or a polycation. Such polyanions and polycations can advantageously be used to obtain polyelectrolyte multilayer films. That is to say, when the polypeptide according to the invention is a polyanion or a polycation, the compound according to the invention can then be incorporated into the layers of a polyelectrolyte multilayer film.

As used throughout the present application, a “polyanion” refers to a polypeptide comprised of amino acids having negatively charged side chains (Asp and/or Glu). A “polycation” refers to a polypeptide comprised of amino acids having positively charged side chains (Lys and/or Arg and/or His and/or Orn).

In a specific embodiment, the polypeptide may for example be comprised of identical amino acids, Examples of such polypeptides include poly(glutamic acid) polypeptides (PGA) (i.e. a polyanion), poly(aspartic acid) polypeptides (i.e. a polyanion), poly(lysine) polypeptides (i.e. a polycation), poly(arginine) polypeptides (i.e. a polycation), poly(histidine) polypeptides (i.e. a polycation) and poly(ornithine) polypeptides (i.e. a polycation). Such polypeptides comprised of identical amino acids can easily be synthesized through chemical synthesis.

The polypeptides according to the invention may be comprised either of amino acids in d form, or of amino acids in l form, or of a mixture thereof.

In a preferred embodiment, the polypeptide according to the invention is a poly(t-glutamic acid) polypeptide, preferably of about 90 to about 120 amino acids. The polypeptide according to the invention can also be a poly(l-glutamic acid) polypeptide having a molecular weight within a range of 15 to 30 Da, preferably about 15, 20, 25 or 30 kDa. Such a polypeptide can for example be obtained through Sigma (St Quentin, France), by buying a composition comprising poly(t-glutamic acid) polypeptides having a molecular weight within a range of 15 to 30 Da, as was done in the Examples.

The α-MSH peptide is coupled to the polypeptide consisting of a chain of about 15 to about 400 amino acids through a covalent link.

Preferably, the polypeptide is coupled to the N-terminal extremity of the α-MSH peptide. As a consequence, the C-terminal extremity of the α-MSH peptide, which is important for the anti-inflammatory activity, is necessarily accessible.

The coupling may be done through any type of chemistry, provided the coupling results in a covalent link. The coupling may for example be done using a maleimide function or using a thiol function.

Pharmaceutical and Veterinary Uses of the Compounds According to the Invention

As set forth hereabove, the compounds according to the invention are for use in endodontic regeneration and/or in the treatment of dental inflammatory diseases. Indeed, the compounds according to the invention allow both controlling the pulp inflammation after infection or injury of a tooth, and promoting regeneration of pulp fibroblasts.

Therefore, the present invention provides a method for endodontic regeneration and/or for treating a dental inflammatory disease comprising the step of administering a effective amount of a compound according to the invention, as defined in the above paragraph, to an individual in need thereof.

Prior to administration of the compound according to the invention, the method according to the invention may comprise the step of cleaning the diseased tooth or lesion, e.g. by removing damaged pulp tissues.

The compound according to the invention may for example be administered through a topical application on the diseased tooth or lesion to be treated. It may also be administered through an injection into the diseased tooth or lesion to be treated.

The compound according to the invention may be applied directly onto the pulp. It may also be applied onto the dentin. Indeed, the compound according to the invention will then spread through the dentin and reach the pulp.

By “effective amount” is meant an amount sufficient to achieve a concentration of peptide which is capable of preventing or treating the disease to be treated. Such concentrations can be routinely determined by those of skill in the art. The amount of the compound actually administered will typically be determined by a physician, in the light of the relevant circumstances, including the condition to be treated, the chosen route of administration, the actual compound administered the age, weight, and response of the individual patient, the severity of the patient's symptoms, etc. it will also be appreciated by those skilled in the art that the dosage may be dependent on the stability of the administered compound.

By “individual in need thereof” is meant an individual suffering from or susceptible of suffering from the disease to be treated.

The term “method of treating” encompasses both therapeutic methods (i.e. aiming at curing, improving the condition and/or extending the lifespan of an individual suffering from the disease) and prophylactic methods (i.e. aiming at preventing the appearance the disease).

As used herein, the term “regenerative endodontics” refers to the replacement of damaged dental structures, including dentin, root structures and cells of the pulp-dentin complex (see e.g. Murray et al. J Endod. 2007 33:377-90 for a review on regenerative endodontics). The damaged dental structure may for example be a diseased, a missing, a necrotic, a traumatized or an infected structure. In the frame of the present invention, the damaged dental structure preferably is damaged pulp.

Regenerative endodontics is useful for treating individuals that suffer from dental inflammatory diseases.

As used herein, the term “dental inflammatory disease” refers to a disease affecting the gum and/or the teeth, wherein said disease is associated with an inflammation of dental tissues such as the gum or the pulp. In the frame of the present invention, the dental disease preferably affects the pulp of the teeth.

Dental inflammatory diseases include but are not limited to pulpal diseases (e.g. pulpitis), dental caries, endodontic lesions, periradicular lesions, gingivitis, peridontitis, periimplantitis, and ulceration (e.g. ulceration due to tissue and/or bone prosthesis).

In a preferred embodiment according to the invention, the dental inflammatory disease is a pulpitis, including reversible, irreversible and chronic pulpitis. Pulpitis is inflammation of the dental pulp resulting for example from untreated caries, trauma, or multiple restorations. Its principal symptom is pain. Diagnosis is based on clinical findings and is confirmed by x-ray. Pulpitis begins as a reversible condition in which the tooth can be saved by a simple filling. It becomes irreversible as swelling inside the rigid encasement of the dentin compromises circulation, making the pulp necrotic, which predisposes to infection. Irreversible and chronic pulpitis currently need to be treated by doing root canal therapy or by extracting the tooth. However, the compounds according to the invention, by regenerating the pulp, now allow avoiding root canal therapy and/or tooth extraction.

The compounds according to the invention find use in the field of human medicine, e.g. when treating one of the dental inflammatory diseases defined hereabove. Therefore, in one embodiment according to the invention, the individual to be treated is a human individual.

The compounds according the present invention also find use in the field of veterinary medicine. Indeed, non-human animals also suffer from dental inflammatory diseases. In particular, since domestic animals attain higher ages than in nature, they often suffer from dental inflammatory diseases such as those defined hereabove.

Therefore, in another embodiment according to the invention, the individual to be treated is a non-human individual, preferably a non-human mammal, most preferably a domestic mammal such as e.g. a cat, a dog, a horse, a cow, a sheep, a goat, a pig, a rabbit, a guinea pig, a hamster, a mouse or a rat.

In a preferred embodiment, the individual to be treated is a cat.

Indeed, cats often suffer from feline odontoclastic resorptive lesions (FORL). FORL is a disease in cats characterized by resorption of the tooth by odontoclasts, cells similar to osteoclasts. A FORL is also known as a neck lesion, cervical neck lesion, cervical line erosion, feline caries, or feline cavity. It is one of the most common diseases of domestic cats, affecting up to two-thirds. FORLs have been seen more recently in the history of feline medicine due to the advancing ages of cats, but 500 year old cat skeletons have shown evidence of this disease. Purebred cats, especially Siamese and Persians, may be more susceptible. FORLs appear as erosions of the surface of the tooth at the gingival border. They are often covered with calculus or gingival tissue. It is a progressive disease, usually starting with loss of cementum and dentin and leading to penetration of the pulp cavity. Resorption continues up the dentinal tubules into the tooth crown. The enamel is also resorbed or undermined to the point of tooth fracture. Resorbed cementum and dentin is replaced with bone-like tissue. Accordingly, FORL is a preferred dental inflammatory disease that can be treated in accordance with the invention.

Pharmaceutical Compositions for Use According to the Invention

The invention also pertains to pharmaceutical compositions comprising the compound according to the invention and a pharmaceutically acceptable carrier, said pharmaceutical compositions being formulated in a manner suitable for use in endodontic regeneration and/or in the treatment of a dental inflammatory disease. More specifically, the present invention provides a method for endodontic regeneration and/or for treating a dental inflammatory disease comprising the step of administering an effective amount of such a pharmaceutical composition to an individual in need thereof.

Pharmaceutical compositions formulated in a manner suitable for use in endodontic regeneration include, e.g., films (also referred to as membranes), toothpastes, varnishes, gels (in particular hydrogels), capsules and solutions (intended for injections or intended for mouth bathes).

The choice of the formulation depends on the intended way of administration. For example, a pharmaceutical composition intended for a topical application, by a skilled practitioner, on the tooth or lesion to be treated may for example be a film, a membrane, a varnish or a gel. A pharmaceutical composition intended for injection, by a skilled practitioner, into the tooth or lesion to be treated preferably is a solution. A pharmaceutical composition intended a topical application by the patient itself (or by the owner of the non-human animal in case of a veterinary use) may for example be toothpaste, a mouth bath or a gel.

As apparent from the above paragraphs, the pharmaceutical composition according to the invention is preferably intended either for a topical application or for an administration by injection, preferably after a chirurgical cleaning of a diseased teeth or lesion to be treated.

In addition to the compound according to the invention, the pharmaceutical composition may comprise other active principles useful for treating dental diseases, such as e.g. an active principle having anti-bacterial activity.

In a specific embodiment according to the invention, the pharmaceutical composition is a gel, preferably a hydrogel. Such hydrogels can for example comprise polysaccharides like chitosan, hyaloronan alginate, cyclodextrin or collagen, optionally mixed with PGA-PEG or DGL-PEG.

In another specific embodiment according to the invention, the pharmaceutical composition is a nanostructured composition. By “nanostructured composition” is meant a structure in which the distribution of the different components is not random.

Such nanostructured compositions are particularly advantageous for use in regenerative endodontics since they provide a “reservoir” of compounds according to the invention, the latter being delivered progressively to the individual to be treated.

Such a nanostructured composition may for example be a polyelectrolyte multilayered film. It may also be a composition (e.g. a film, a gel or a varnish) comprising nanoparticles, capsules or polymers to which the compound according to the invention can bind (e.g polyanions, polycations, DGLs, etc.).

Such a nanostructured composition may for example comprise calcium phosphate nanoparticles (e.g. as those described in Zhang et al., Biomaterials, 2010, 31:6013-8). Such nanoparticles are believed to be especially suitable for use in regenerative endodontics since calcium phosphate is a natural component of dentin. Alternatively, it may comprise polycation or polycation nanoparticles (e.g. a poly(lysine) nanoparticle as described in Zhang et al. Biomaterials, 2010, 31:1699-706) or a capsule (preferably a multilayered capsule, e.g. as that described in Facca et al. Proc Natl Acad Sci USA., 2010, 107:3406-11).

In a specific embodiment, the capsules and/or nanoparticles are used as a coating on an implantable material such as a prosthesis.

In a most preferred embodiment according to the invention, the pharmaceutical composition is a polyelectrolyte multilayered film.

Polyelectrolyte multilayered films are composed of at least one layer pair consisting of a layer of polyanions and of a layer of polycations. They may for example comprise 1, 2, 3, 4, 5, 6, 7, 8, 9, 10, 11, 12, 13, 14, 15 or more layer pairs. Preferably, it comprises from 5 to 12 layer pairs.

Polyelectrolyte multilayered films can easily be obtained by the alternate dipping of a charged surface in polyanion and polycation solutions. The simplicity and versatility of such build-up procedure makes them a pharmaceutical composition of choice.

In addition, bioactive molecules can be incorporated into polyelectrolyte multilayered films. Examples of such polyelectrolyte multilayered films that comprise a bioactive molecule are disclosed in WO 02/085423 and WO 2006/079928.

The polyelectrolyte multilayered films preferably comprise or consist of at least 1, 2, 3, 4, 5, 6, 7, 8, 9, 10, 11, 12, 13, 14, 15 or more layer pairs, each layer pair consisting of:
a layer of polyanions selected from the group consisting of poly(glutamic acid) polypeptides (PGA), poly(aspartic acid) polypeptides and a compound according to the invention, provided said compound according to the invention comprises a polypeptide being a polyanion; and
a layer of polycations selected from the group consisting of poly(lysine) polypeptides, poly(arginine) polypeptides, poly(histidine) polypeptides, poly(ornithine) polypeptides, Dendri-Graft Poly-lysines (e.g. Dendri-Graft lysines) and a compound according to the invention, provided said compound according to the invention comprises a polypeptide being a polycation

The cationic and anionic polypeptides comprised within the polyelectrolyte multilayered film may be comprised either of amino acids in d form, or of amino acids in l form, or of a mixture thereof.

The polyelectrolyte multilayered films according to the invention necessarily comprise a compound according to the invention. The compound according to the invention may for example be adsorbed on the top of the polyelectrolyte multilayered film. This can be done by bringing the compound according to the invention in contact with a polyelectrolyte multilayered film terminating with positively charged layer (see Example 1). Alternatively, the compound according to the invention may be embedded within the polyelectrolyte multilayered film, preferably at each layer pair of said film. Such a polyelectrolyte multilayered film can for example be obtained by using the compound according to the invention as a polyanion when building the film (see Example 1).

In a preferred embodiment, the polyelectrolyte multilayered film comprises at least one layer pair consisting of:
a layer of poly(lysine) polypeptides and a layer of poly(glutamic acid) polypeptides; and/or
a layer of poly(lysine) polypeptides and a layer of compounds according to the invention; and/or
a layer of Dendri-Graft Poly-lysines (e.g. Dendri-Graft Poly-L-lysines) and a layer of compounds according to the invention,
   the amino acids of the polypeptides being either amino acids in d form, or amino acids in l form, or a mixture thereof.

In a specific embodiment, the polyelectrolyte multilayered film is one of those described in the examples, i.e. (PLL-PGA-α-MSH)₁₀, (DGL^{G4}-PGA-α-MSH)₁₀, or (PLL-PGA)₅-PLL-PGA-α-MSH. “PGA” stands for Poly-l-glutamic acid, “PLL” stands for Poly-l-lysine, and “DGL^{G4}” stands for fourth-generation Dendri-Graft Poly-L-lysines. “ₙ” indicates the number of layer pairs. In (PLL-PGA)₅-PLL-PGA-α-MSH, the compound according to the invention is adsorbed on the top of the polyelectrolyte multilayered film. In (PLL-PGA-α-MSH)₁₀ and (DGL^{G4}-PGA-α-MSH)₁₀, the compound according to the invention is embedded within the polyelectrolyte multilayered film.

Pharmaceutical Compositions Comprising DGLs

In the present invention, the inventors describe for the first time a composition comprising α-MSH-PGA associated with DGLs. DGLs form reticulated structures to which the compound according to the invention can bind. DGLs are thus particularly suitable for use as a “reservoir” for the compounds according to the invention.

Therefore, the present invention provides a pharmaceutical composition comprising a compound according to the invention and Dendri-Graft Poly-lysines, preferably Dendri-Graft Poly-L-lysines (DGLs).

In the frame of this aspect of the invention, the compound according to the invention preferably comprises a polypeptide being a polyanion, such as e.g. a poly(glutamic acid) polypeptide or a poly(aspartic acid) polypeptide.

Methods for obtaining DGLs are known to the skilled in the art, DGLs can for example be prepared as described in Example 1 and/or in Colletet et al., (Chem. Eur. J. 2010, 16:2309-2316).

According to the invention, the DGLs can be of any generation, e.g. be of first, second, third or fourth generation. DGLs of first generation have a DPn of 8 and a polydispersity of 1.2. DGLs of second generation have a DPn of 48 and a polydispersity o 1.3. Third generation DGLs have a DPn of 123 and a polydispersity of 1.4. Fourth generation DGL have a DPn of 365 and a polydispersity of 1.3. Preferably, the DGLs according to the invention are of fourth generation (DGL^{G4}).

Such a pharmaceutical composition can for example be a polyelectrolyte multilayered film, a gel (in particular a hydrogel), a varnish, a solution or toothpaste.

In a preferred embodiment, the pharmaceutical composition comprising a compound according to the invention and DGLs is a polyelectrolyte multilayered film that comprises at least 1, 2, 3, 4, 5, 6, 7, 8, 9, 10, 11, 12, 13, 14, 15 or more layer pairs consisting of a layer of Dendri-Graft Poly-L-lysines (DGLs) and a layer of compounds according to the invention.

In a most preferred embodiment, the pharmaceutical composition comprising a compound according to the invention and DGLs is a (DGL^{GA}-PGA-α-MSH)₁₀ polyelectrolyte multilayered film.

All references cited herein, including journal articles or abstracts, published patent applications, issued patents or any other references, are entirely incorporated by reference herein, including all data, tables, figures and text presented in the cited references.

Although having distinct meanings, the terms “comprising”, “having”, “containing’ and “consisting of” may be replaced with one another throughout the above description of the invention.

In the frame of the present description, all compounds, polypeptides and peptides may optionally be isolated and/or purified.

The invention will be further evaluated in view of the following examples and figures.

### BRIEF DESCRIPTION OF THE FIGURES

FIG. 1: Kinetics of TNF alpha □(upper part), IL-8 (Middle part) and IL-10 Lower part) production by LPS-stimulated pulp fibroblasts. Cells were incubated at a density of 5×10⁵ cells/well in the presence of 10 ng·mL⁻¹ of LPS and 100 μg·mL⁻¹ of free α-□MSH or of PGA-α-MSH for 20 min, 1 h, 2 h, 4 h and 6 h: TNF-alpha □production (Upper part) IL-8 production (Middle part), and IL-10 production (Lower part). Clear bars represent the secretion with LPS alone, black bars represent the values obtained when cells were incubated with LPS+free α-MSH, and hatched bars represent the values obtained after incubation with LPS+PGA-α-MSH. An average of three measurements with the corresponding, standard error is shown. *P<0.05 vs. LPS alone

FIG. 2: Kinetics of IL-8 production by pulp fibroblasts. Cells were incubated at a density of 5×10⁵ cells/well in the absence of LPS. IL-8 production by human pulp (Upper part) and NIH 3T3 (Lower part) fibroblasts was measured after 0 min, 1 h, 2 h, 4 h and 6 h of cell contact with different concentrations of PGA-α-MSH (0 μg·mL⁻¹: black bars; 25 μg·mL⁻¹□: grey bars; 50 μg·mL⁻¹: □hatched bars; 75 μg·mL⁻¹□: dotted bars; 100 μg·mL¹: clear bars). An average of three measurements with the corresponding standard error is shown. *P<0.05 vs. control (PGA).

FIG. 3: Intracellular cAMP accumulation by pulp fibroblasts: Pulp fibroblasts were incubated with (a): vehicle (negative control), (b): Forskolin: 3 μM, (c): MTII: 10 μg·mL-1, (d): MS05: 10 μg·mL-1, PGA-α-MSH (e): 10 μg·mL-1, (f): 30 μg·mL-1, (g): 100 μg·mL-1 or (h): 300 μg·mL-1 and cell-associated cAMP determined at the 30 min time-point. Cell incubation with forskolin (used as a positive control) led to 3367±242 fmol/well (n=6). Data expressed as mean±SEM of three experiments. *P<0.05 vs. vehicle (a).

FIG. 4: Viability and proliferation of pulp fibroblast. Upper part: Viability of pulp fibroblast after one day (D1), two days (D2) or 4 days (D4), the percentage of viable cells has been calculated as followed: number of viable cells×100/number of total cell with an average of eight measurements with the corresponding standard error. Lower part: Cells proliferation has been checked by the acid phosphatase assay for quantifying the growth of adherent and non-adherent cells. The background corresponds to an absorbance of 0.16 at λ=405 nm. An average of three measurements with the corresponding standard error was represented

FIG. 5: Pulp fibroblasts morphological changes after 2-days of culture: Pulp fibroblasts were cultivated in the presence of α-MSH or PGA-α-MSH. After 2 days, the Quantification of the adherent cells in the presence of α-MSH or PGA-α-MSH in solution were analyzed.

FIG. 6: Pulp fibroblasts morphological changes after 2-days of culture: Cell-morphology and quantification of adherent cells of pulp fibroblasts after 2 days of culture seeded on the multilayered films (PLL-PGA)₅-PLL-PGA (left panel) or (PLL-PGA)₅-PLL-PGA-alpha-MSH (middle panel). Quantification of adherent cells on the LBL films is shown (right panel).

FIG. 7: Proliferation and morphology of pulp fibroblasts growing on the surface of the multilayered films. Proliferation of pulp fibroblast after two days of culture on the surface of the multilayered films (PLL-PGA)₁₀ (A), (PLL-PGA-αMSH)₁₀ (B), (DGL^{G4}-PGA)₁₀ (C) and (DGL^{G4}-PGA-αMSH)₁₀ (D) followed by AlamarBlue. Morphology and actin organization of cells (fixation after 2 days of proliferation) observed by confocal microscopy: Alexa Fluor 546-labelled Phalloidin (red) and DAPI (blue).

FIG. 8: Dissipation versus frequency shift analysis by QCM of the multilayered films during their build-up process. Successive adsorptions on top of a SiO₂-coated quartz crystal sensor were monitored in situ by QCM for (PLL-PGA)₁₀ (), (PLL-PGA-α-MSH)₁₀ (▾), (DGL^{G4}-PGA)₁₀ (▪) and (DGL^{G4}-PGA-α-MSH)₁₀ (♦) multilayered films. Values of frequency shift and dissipation are collected and the linear regressions are added in the graph to follow the build-up of each film. The zoom of the low values of dissipation and frequency was presented as insert.

### BRIEF DESCRIPTION OF THE SEQUENCES

SEQ ID NO: 1 shows the sequence of a human α-MSH peptide.

SEQ ID NO: 2 shows the sequence of a biologically active analogue of the human α-MSH peptide of SEQ ID NO: 1.

### EXAMPLES

### Example 1

### Materials and Methods

Chemicals

The α-MSH analogue, HS—CH₂CH₂-Ser-Tyr-Ser-Nle-Glu-His-_{D}-Phe-Arg-Trp-Gly-Lys-Pro-Val-COOH (i.e. a peptide of SEQ ID NO: 2 coupled to a maleimide function at its N-terminus), was obtained from Neosystem (Strasbourg, France). Poly-l-glutamic acid (PGA), Poly-l-lysine hydrobromide (PLL), N-hydroxysulfosuccinimide, lipopolysaccharide (LPS) from E. coli 026:B6, and phorbol ester 12-O tetra decanoyl phorbol 13 acetate (TPA) were obtained from Sigma (St Quentin, France).

### Synthesis of PGA-α-MSH Films

The α-MSH peptide was covalently coupled to poly-l-glutamic acid (PGA) and used free or incorporated into the polyelectrolyte multilayer films (PLL-PGA)ₙ-PLL as previously described (Jessel et al. Adv. Materials, 2004, 16:1507-1511; Schultz et al., Biomaterials. 2005, 26:2621-2630).

### Synthesis of Dendri Graft Poly-L-Lysines (DGLs)

DGLs were prepared as described in Colletet et al. (Chem. Eur. J. 2010, 16:2309-2316). In brief, Ne-TFA-_{L}-lysine-NCA, prepared according to a method reported in Collet et al., (Tetrahedron Lett. 1996, 37:9043-9046), was dissolved in an aqueous H₂—CO₃/HCO₃Na solution (pH 6.5) (Commeyras et al. Polym. Int. 2002, 51:661-665), leading, after 30 min, to oligo-Ne-TFA-_{L}-lysine, which precipitates and is isolated by filtration. The protecting group is removed with 150 mL of water-methanolammonia solution at pH 11 (15 h at 40° C.). Following partial solvent removal at reduced pressure, the remaining solution was freeze-dried, affording a first-generation oligo(L-lysine) of DPn=8 and low polydispersity (1.2). By repeating the same polymerization procedure in the presence the first-generation oligo(L-lysine), second-generation DGL (DGL^{G2}) was obtained (DPn=48 and Polydispersity=1.3) following deprotection and freeze-drying. The third-generation DGL (DGL^{G3}) was obtained (DPn=123 and polydispersity=1.4) under the same conditions using DGL^{G2}. And so on, the fourth-generation DGL (DGL^{G4}) was obtained (DPn=365 and polydispersity=1.3) under the same conditions using DGL^{G3}. The structures of DGL^{G2}, DGL^{G3} and DGL^{G4} were established by ¹H and ¹³C NMR. In this study, we have used the fourth-generation DGL^{G4}.

Polyelectrolyte Multilayered Film Preparation

For all biological activity experiments, polyelectrolyte multilayer films were prepared on glass cover slips (CML, France) and pre-treated with 10⁻² M SDS and 0.12 N HCl for 15 min at 100° C., and then extensively rinsed with deionised water. The glass cover slips were then deposited in 24 well plates (Nunc, Denmark). A precursor film constituted by PLL-(PGA-PLL) was built by alternating immersion of the plates during 20 min in the respective polyelectrolyte solutions (300 ml) at the respective concentrations of 1 mg/ml for PGA and PLL in presence of 0.15M NaCl at pH=7.4. PGA-α-MSH is negatively charged and was always adsorbed on a film terminating with a layer of positively charged PLL. After each deposition step the cover slips were rinsed three times during 5 min with deionized water. All the films were sterilized for 10 min by exposure to ultraviolet (UV) light (254 nm, 30 W, illumination distance 20 cm). Before use, all films were equilibrated in contact with 1 ml of RPMI medium (see Cell culture) without serum.

Cell Culture

Human pulp fibroblasts were obtained from human third molar germ tissue extracted for orthodontic reasons (Dental School, Strasbourg). These tissues were used with the patient's informed consent and approval by the Research Ethics Committee. Human pulp fibroblasts and mouse embryo fibroblasts (NIH 3T3, a gift of IGBMC, Strasbourg, France) were cultured in Dulbecco's modified Eagle's medium (D-MEM®) containing 1% antimicotic solution and 10% FBS (Life Technologies, Paisley, UK). The cultures were incubated at 37° C. in a humidified atmosphere of 5% CO2. When the cells reached sub-confluence, they were harvested with trypsin and sub-cultured. The fibroblast cultures used for all experiments were from passages three to six.

Cell Viability and Proliferation

Cell viability was determined by trypan blue exclusion. Cell proliferation was measured by the acid phosphatase method. Briefly, cells were washed with 300 mL PBS. The buffer added to each well contains 0.1 M sodium acetate (pH 5.5), 0.1% of X-100 Triton and 10 mM of p-nitrophenyl phosphate [pNPP] (Sigma, St Quentin, France). After a 3 h-incubation at 37° C. with 5% 002 the reaction was stopped by the addition of 1 N sodium hydroxide and the absorbance was measured by spectrophotometer at 405 nm (Labsystems, iEMS Reader MF, Gibco). Cell viability and proliferation were measured at 24 and 96 hours. Cell spreading and adhesion were observed by confocal microscopy. AlamarBlue® (Serotec) was used to assess cellular proliferation. The AlamarBlue test is a non-toxic, water-soluble, calorimetric redox indicator that changes color in response to cell metabolism. In this study, 5×10⁴ pulp fibroblasts were seeded on the top of LbL-coated 14 mm-diameter coverslips (n=3) placed on 24-well plates. After 2 days of culture, cells were incubated in 10% AlamarBlue/DMEM solution in a humidified atmosphere at 37° C. and 5% CO2. After 6 hours, 100 mL of incubation media was transferred to 96-well plates and measured at 590 nm and 630 nm in order to determine the percentage of AlamarBlue reduction.

Immunofluorescence

After 2 days of proliferation, cells were fixed with 4% PFA during 1 hour, permeabilized with 0.1% Triton X-100 for 1 hour and incubated for 20 min with Alexa Fluor 546-conjugated phalloidin (Molecular Probes) for F-actin labeling and 5 min with 200 nM DAPI (Sigma) for nuclear staining. Cells were mounted on microscope slides using Vectashield (Vector) and imaged by confocal microscopy (Zeiss, LSM 510).

Cell Activation

Fibroblasts (5×10⁵) were seeded in 24-well plates (Nunc, CML, Nemours, France) and incubated with LPS (10 ng·mL⁻¹) from E, coli in the presence or absence of free α-MSH or PGA-Q-MSH coupled peptides, in D-MEM® without FBS. At different time-points of incubation, cells were centrifuged and the levels of TNF-α, IL-10 or IL-8 in cell-free supernatants were determined using commercially available ELISA kits (Endogen, Woburn, Mass.). Studies of the effects of multilayer films consisting of PGA-α-MSH coupled peptides were conducted by seeding 5×10⁵ cells directly onto the film in a 24-well plate as previously described (Chluba et al. Biomacromolecules. 2001, 2:800-805). All experiments were performed at least three times.

Intracellular cAMP

Pulp fibroblasts (1×10⁵) were plated in 24-well-plates in D-MEM® containing 1% antimicotic solution and 10% FBS (Life Technologies, Paisley, UK). The cultures were incubated at 37° C. in a humidified atmosphere of 5% CO, and allowed to adhere for 2 h. Cells were then washed and incubated with 30M Forskolin (positive control), the MC3/4R agonist MTII (10 μg·mL⁻¹), the MC1R agonist MS05 (10 μg·mL⁻¹), and PGA-α-MSH (10-300 μg·mL⁻¹) in the presence of 1 mM isobutylmethylxantine in serum-free medium and incubated for 30 min at 37° C. in a humidified atmosphere of 5% CO2. A non-treated group incubated in serum-free medium alone served as a negative control. Cell supernatants were then removed and the cells were lysed. Intracellular cAMP was quantified using a commercially available enzyme immunoassay kit and a standard curve constructed with 0-3200 fmol μg·mL-1 cAMP (Amersham Bioscience, Little Chalfont, U.K).

Quartz Crystal Microbalance

The films were monitored in situ with a quartz crystal microbalance using an axial flow chamber QAFC 302 (QCM-D, D300, Q-Sense, Götenborg, Sweden). QCM works by measuring the resonance frequency shift (Δf) of a quartz crystal induced by polyelectrolyte or protein adsorption onto the crystal in comparison to the crystal in contact with buffer. Changes in the resonance frequencies were measured at the third overtone (n=3), corresponding to the 15 MHz resonance frequency. A shift in Δf/n can be related in a first approximation to a variation of the mass adsorbed to the crystal by the Sauerbrey relation: m=−C Δt/n, where C is a constant characteristic of the crystal used (in our case: C=17.7 ng cm⁻² Hz⁻¹). The qualitative information about the viscoelastic properties of the film can be analyzed by using the ratio (R):ΔD/(f/v). An increasing value of this ratio indicates a decreasing of stiffness of the deposited material.

Atomic Force Microscopy

The images were obtained in contact mode in liquid conditions with the Solver Pro from NT-MDT (Moscow, Russia). Cantilevers with a spring constant of 0.03 N/m and with silicon nitride tips were used (Model MSCT-AUHW Park Scientific, Sunnyvale, Calif., USA). Several scans were performed over a given surface area. These scans had to give reproducible images to ascertain that there is no sample damage induced by the tip. Deflection and height mode images are scanned simultaneously at a fixed scan rate (between 2 Hz) with a resolution of 512×512 pixels. For all the observations, the samples were kept under liquid (build-up buffer)

Confocal Laser Scanning Microscopy (CLSM)

CLSM observations were documented with a Zeiss LSM 510 microscope using a ×40/1.4 oil immersion objective at 0.4 μm z-section intervals. FITC fluorescence was detected after excitation at 488 nm with a cutoff dichroic mirror 488 nm and an emission band-pass filter 505-530 nm (green). All experiments were performed with cells in solution.

Statistical Analysis

All values are expressed as mean±SEM and all experiments were repeated at least three times. Statistical analysis was performed using the Mann Whitney U test. A probability p value<0.05 was considered significant to reject the null hypothesis.

### Example 2

### Effects of α-MSH and PGA-α-MSH on Production of TNF-α, IL-10, and IL-8 by LPS-Stimulated Pulp Fibroblasts

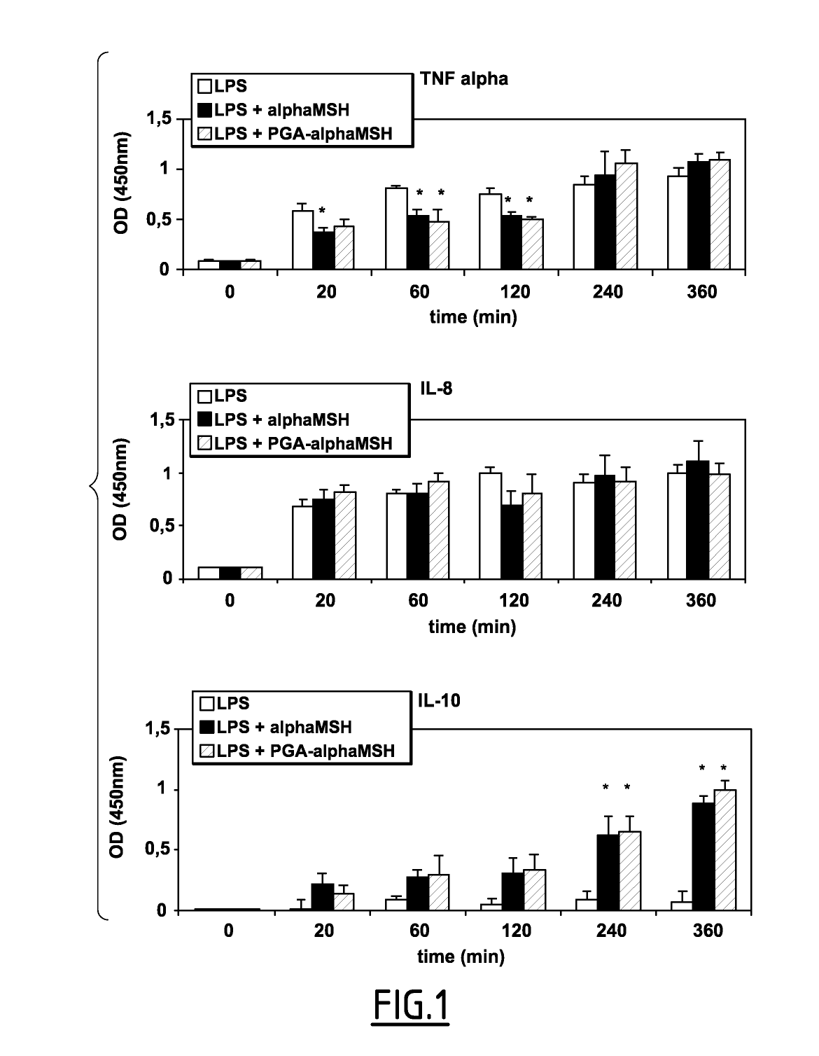

Pulp fibroblasts were stimulated with LPS (10 ng mL⁻¹) in the presence of either 100 μg·mL-1 free α-MSH or PGA-α-MSH. The amount of TNF-α secreted by cells in the presence of LPS was significantly increased over a 6 h time period (FIG. 1, upper part). At early timepoints (20 min to 2 h), however, α-MSH and PGA-α-MSH significantly inhibited the amount of LPS induced TNF-α secretion, while at later time-points (4-6 h) this effect was lost. In contrast, no inhibition of LPS-induced IL-8 secretion was observed in the presence of either 100 μg·mL⁻¹ α-MSH or PGA-α-MSH during this 6 h time period (FIG. 1, middle part). Furthermore, increasing the concentrations of both α-MSH and PGA-α-MSH 400 μg·mL-1 did not change the amount of the IL-8 produced (data not shown). In contrast to these observations, both α-MSH and PGA-α-MSH induced significant levels of the anti-inflammatory cytokine, IL-10 (FIG. 1, bottom part).

### Example 3

### Effects of PGA-α-MSH on IL-8 Production by Pulp Fibroblasts in the Absence of LPS

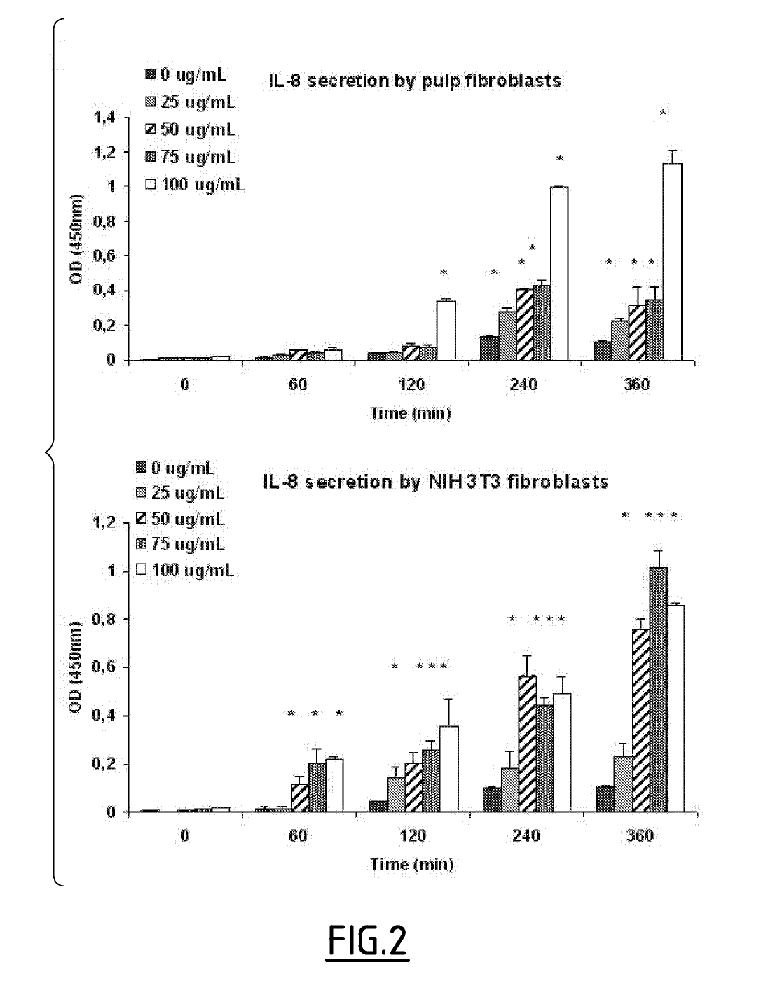

Previous studies have shown that α-MSH stimulates the production of IL-8 by dermal fibroblasts. To characterize the ability of PGA-α-MSH to stimulate IL-8, the levels of IL-8 expression were determined in LPS-free fibroblast cultures. Fibroblasts (pulp, FIG. 2, upper part and NIH 3T3, FIG. 2, lower part) stimulated with PGA-α-MSH showed a time and concentration-dependent increase in IL-8 production, with NIH 3T3 fibroblasts being more sensitive than primary pulp fibroblasts. The first significant increase in IL-8 could be detected with 50 μg·mL⁻¹ of PGA-α-MSH on NIH 3T3 fibroblasts and with 100 μg mL⁻¹ of PGA-α-MSH on pulp fibroblasts after 1 h and 2 h of contact, respectively.

### Example 4

### PGA-α-MSH Internalization and Effects on cAMP Accumulation in Pulp Fibroblasts

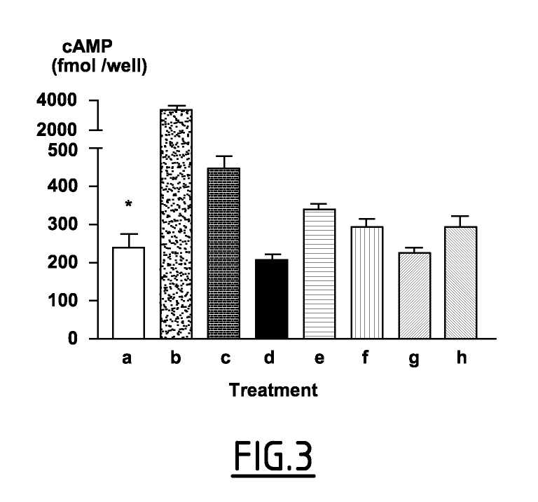

Since α-MSH peptides are most effective when internalized by monocytes via their receptors, the degree of internalization of PGA-α-MSH by pulp fibroblasts was assessed. Incubation of pulp fibroblasts with FITC-labelled PGA-α-MSH (100 μg·mL⁻¹) resulted in a homogenous fluorescence of the fibroblasts after 2 h, indicating internalization (data not shown). The effect of PGA-α-MSH on cAMP accumulation in pulp fibroblasts was then determined to ascertain whether it exerted its biological effects via activation of MCR receptors. Both the positive control, forskolin, and the non-selective agonist MTII, significantly increased cAMP concentrations to 3367±242 and 459±14 fmol/well, respectively, above the basal level of 239±36 fmol/well. In contrast, both the selective MC1R agonist (MS05) and PGA-α-MSH failed to significantly increase CAMP accumulation at any of the concentrations tested (FIG. 3).

### Example 5

### Effects of PGA-α-MSH on Pulp Fibroblast Proliferation and Adhesion

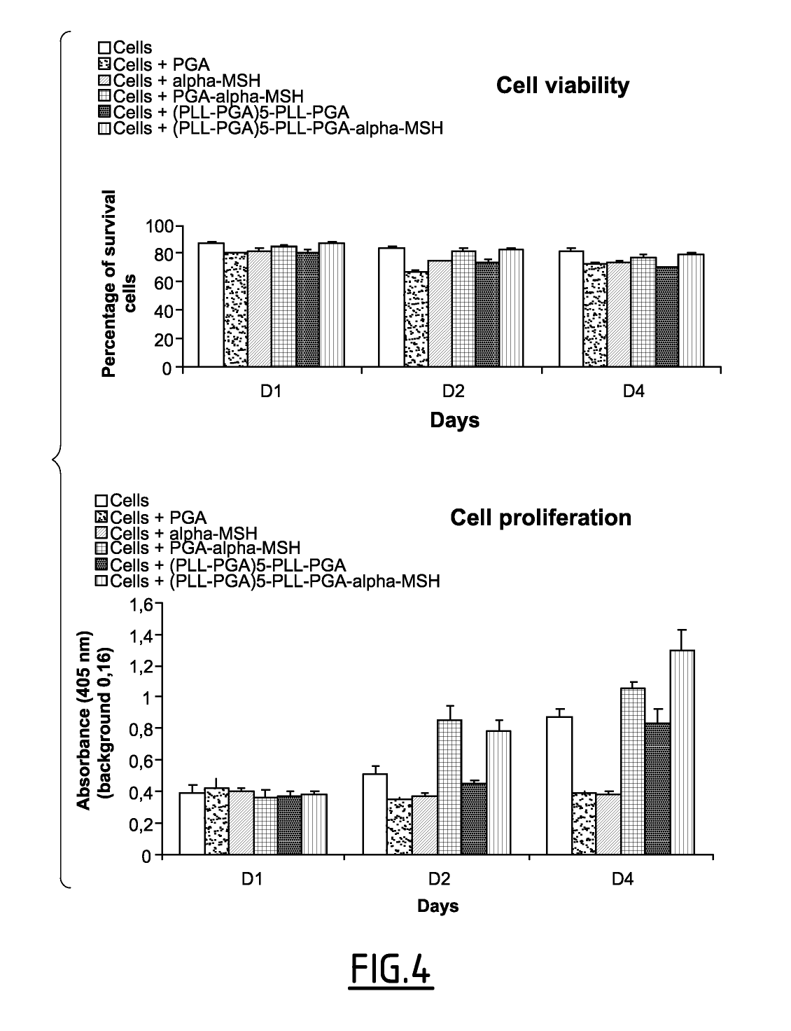

The ability of free α-MSH and PGA-α-MSH to promote proliferation and/or adhesion of fibroblasts was also determined. Cell viability was determined by trypan blue exclusion and the proliferation was measured by the acidic phosphatase method (FIG. 4).

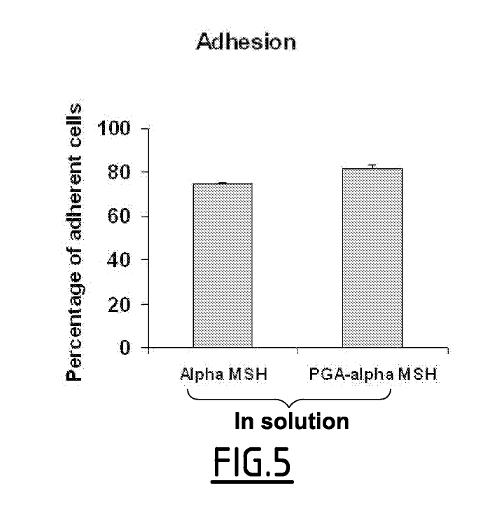

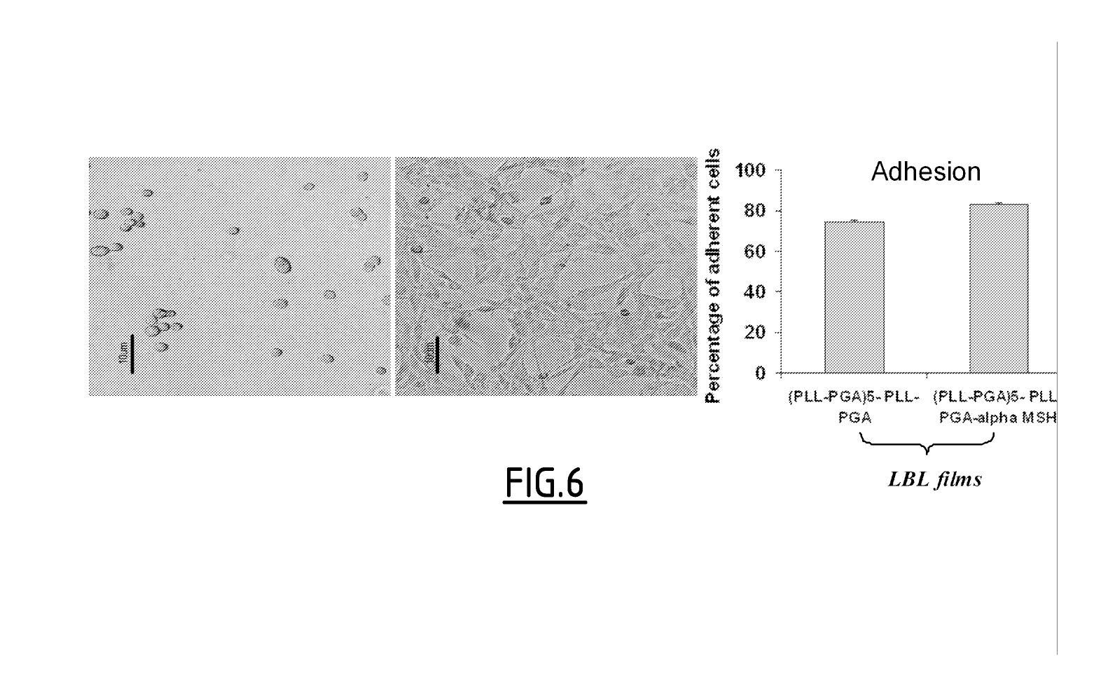

We have also shown by confocal microscopy that the incubation of fresh seeded pulp fibroblasts with α-MSH, PGA-α-MSH in solution indicate clearly that while α-MSH inhibits the proliferation of the cells (FIG. 4, lower part) (but not the viability, FIG. 4, upper part), the presence of α-MSH covalently coupled to PGA [PGA-α-MSH and (PLL-PGA)₅-PLL-PGA-α-MSH] promotes adhesion (FIG. 5). On the multilayered film, when PGA inhibit the adhesion of cells (FIG. 6, left panel). PGA-α-MSH on multilayer film induce this adhesion (FIG. 6, middle panel).

### Example 6

### Pulp Fibroblast Behaviors Effects of PGA-α-MSH when Incorporated into the Multilayered Film (PLL-PGA-α-MSH)₁₀ by Using PLL or the Dendri Graft Poly-L-Lysines (DGLs)

In this study, we analyzed the effect of PGA-α-MSH on pulp fibroblast when PGA-α-MSH is not only adsorbed on the top of the multilayered films (see FIG. 6) but incorporated at each level as a reservoir for cells (PLL-PGA-α-MSH)₁₀ or by using the DGL^{G4} (Collet at al. Chem. Eur. J. 2010, 16:2309-2316; Collet et al. Tetrahedron Lett. 1996, 37; 9043-9046; Commeyras et al. Polym. Int. 2002, 51:661-665) to be able to adsorb more PGA-α-MSH and to increase the efficiency in term of proliferation effect.

In FIG. 7, we analyzed the Proliferation and morphology of pulp fibroblasts growing on the surface of the multilayered films (PLL-PGA)₁₀ (A), (PLL-PGA-α-MSH)₁₀ (B), (DGL^{G4}-PGA)₁₀ (C) and (DGL^{G4}-PGA-α-MSH)₁₀ (D) followed by AlamarBlue. We have also analyzed by confocal microscopy morphology and actin organization of these cells after 2 days of proliferation (data not shown).

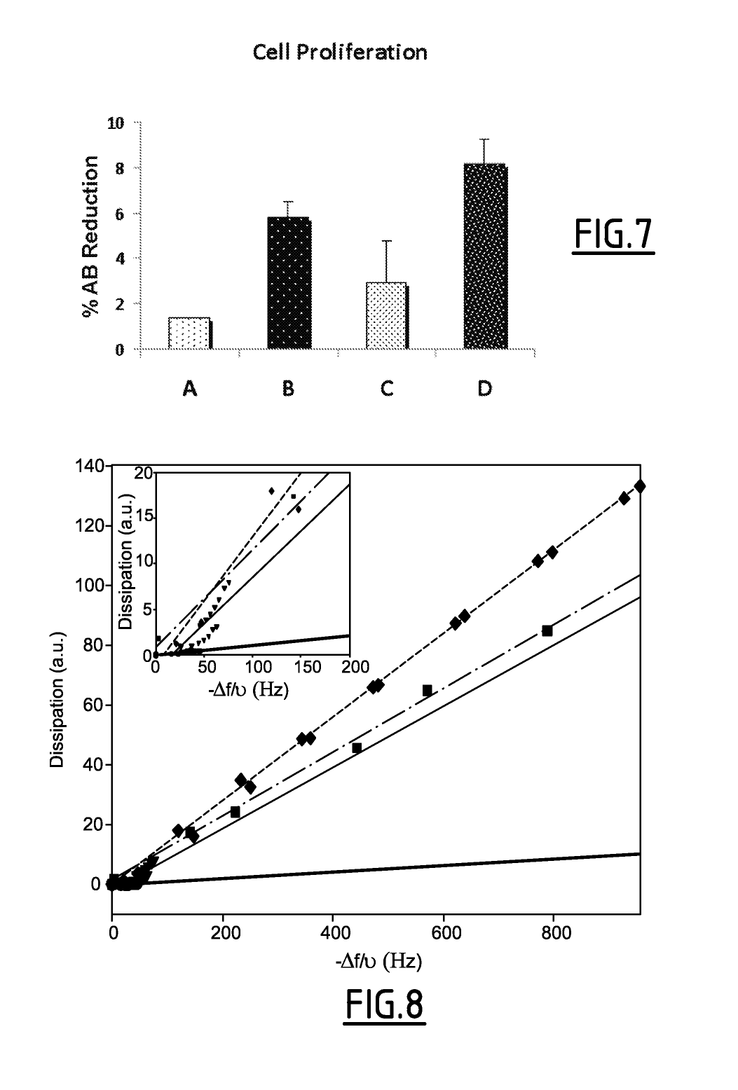

First we analyzed by Quartz crystal microbalance (QCM) dissipation versus frequency shift of the multilayered films during their build-up process. Successive adsorptions on top of a SiO₂-coated quartz crystal sensor were monitored in situ by QCM for (PLL-PGA)₁₀ (A), (PLL-PGA-α-MSH)₁₀ (B), (DGL^{G4}-PGA)₁₀ (C) and (DGL^{G4}-PGA-α-MSH)₁₀ (D) multilayered films. In FIG. 8, the Values of frequency shift and dissipation are collected and the linear regressions are added in the graph to follow the build.

In the next step, atomic force microscopy (AFM) was used to get additional information about the structure of these multilayered architectures at the nanoscale (data not shown).

Modification of dissipation can be correlated to increase the roughness of films analyzed by Atomic force microscopy (AFM) (Jessel et al. Proc. Nat. Acad. Sci. USA. 2006, 103:8618-8621). In this study, we have also analyzed the roughness and thickness of these nanostructured films (Architectures A, B, C and D) obtained by In situ AFM images height mode in buffer of these films (Table 1).

TABLE 1
Physical Characterization of the Multilayered Films^{a}

| nanostructured | roughness | thickness | ΔD/(f/ν) |
|---|---|---|---|
| film | rms (nm) | (nm) | (QCM) |
| A | 4.1 ± 0.3 | 7.7 ± 2.2 | 0.01 ± 0.0015 |
| B | 66.1 ± 13.6 | 307 ± 48 | 0.10 ± 0.026 |
| C | 2.3 ± 0.7 | 8.2 ± 1.1 | 0.10 ± 0.018 |
| D | 18.7 ± 2.3 | 538 ± 100 | 0.14 ± 0.001 |

^{a}Roughness, thickness and ΔD/(f/ν) analysis of the multilayered films (PLL-PGA)₁₀ (A), PLL-PGA-α-MSH)₁₀ (B), (DGL^{G4}-PGA)₁₀ (C), and (DGL^{G4}-PGA-α-MSH)₁₀ (D) obtained by AFM and QCM.

### Example 7

### Discussion

Melanocortin peptides have been shown to possess anti-inflammatory effects in several experimental models of acute and chronic inflammation and in human cells such as monocytes, astrocytes and keratinocytes. The biological effects of α-MSH are exerted partially via direct binding to their receptors (MCR) resulting in adenylate cyclase-mediated conversion of ATP to cyclic AMP and inhibition of pro-inflammatory cytokines. In addition to these anti-inflammatory properties in various cell types α-MSH has also recently been shown to antagonize the effects of TGF-3 on collagen synthesis by dermal fibroblasts.

In this study, the effects of α-MSH and PGA-α-MSH on pulp fibroblasts was investigated.

To improve implant biocompatibility, polyelectrolyte multilayers on charged surfaces offers an important approach. We have previously developed bioinert materials able to create, through surface modifications, a bioactive interface regulating biological responses (Jessel at al. Adv. Materials, 2004, 16:1507-1511; Jessel et al., Adv. Functional Materials, 2004, 14:174-182; Schultz at al. Biomaterials. 2005, 26:2621-2630; Jessel et al. Adv. Materials, 2003, 15:692-695; Jessel et al. Adv. Functional Materials, 2004, 14:963-969; Jessel et al. Adv. Functional Materials, 2005, 15:648-654; Picart et al. Adv. Functional Materials, 2005, 15:1771-1780 and Gangloff et al. Biomaterials, 2005, 27:1771-1777). In addition, we previously observed in a monocyte model where we examined TNF-α and IL-10 secretions, that PGA-α-MSH induces a response similar to that of α-MSH in solution ((Jessel et al. Adv. Materials, 2004, 16:1507-1511).

As previously observed with monocytes, PGA-α-MSH, in contact with LPS-induced pulp fibroblasts, induces a bimodal anti-inflammatory response with an early inhibition of TNF-α production that is statistically significant after the first and second hour of activation, and a later (4 hours) statistically significant induction of the anti-inflammatory cytokine, IL-10.

These results have a significant impact on pulpal inflammation because TNF-α induce significant high levels of vascular growth factor mRNA gene expression in human pulp which may promote apical expansion of the inflammation (Chu et al., J. Ended. 2004, 30:704-707). Therefore long-term expression of TNF-α may avoid healing process as reactinary or reparative dentin formation witch acts to increase the barrier between the cells of the pulp and the injury (Min at al. J. Endod. 2006, 32:39-43).

The induction of IL-10 by PGA-α-MSH is also crucial because IL-10 inhibit synthesis of most pro-inflammatory cytokines by dental pulp cells (Tokuda et al. J. Endod., 2002, 28:177-180).

Recently, it was reported that inflamed pulps present higher amounts of IL-1 beta and IL-8 than healthy pulps and that pulp fibroblasts stimulated also by Escherichia coli LPS produce higher levels of IL-1-beta and IL-8 than the control group (Silva et al. J. Appl. Oral. Sci. 2009, 17:527-532). However, both α-MSH and PGA-α-MSH were unable to inhibit IL-8 release by LPS-stimulated pulp fibroblasts. The apparent discrepancy with studies in which α-MSH inhibited IL-1 induced IL-8 production in dermal fibroblasts (Bohm et al. Ann. N.Y. Acad. Sc., 1999, 885:277-286) could be due to differing stimuli, LPS versus IL-1, or to the type of fibroblasts used. Even though NF-κB mediated IL-8 gene expression can be induced by LPS through the CD14/TLR4 receptor complex or by IL-1 through the IL-1 receptor, the signalling pathways leading to IL-8 expression are partially different (Yamamoto at al. Nat. Immunol. 2003, 4:1144-1150; O'Neill at al. Trends Immunol. 2004, 25:687-693). Furthermore, the involvement of α-MSH in these pathways is not identical (Sarkar at al. FEBS letters 2003, 553:286-294). In addition, since IL-1 is a better inducer of IL-8 than LPS in fibroblasts, the antagonist effects of α-MSH on IL-8 production might be more striking on IL-1 stimulated than on LPS-stimulated fibroblasts.

Free α-MSH is known to stimulate IL-8 expression by dermal fibroblasts. To ascertain whether PGA-α-MSH retains this capacity, its effects on LPS-free pulp and NIH 3T3 fibroblasts were evaluated. Similar IL-8 secretion profiles were found, confirming that the stimulation more than the cell type is important for free-α-MSH or PGA-α-MSH overall effect. Recently, we and other reported clearly that the PGA-α-MSH kept its full activities when adsorbed on the top of the multilayered polyelectrolyte films by using different kind of cells in term of melanin production or cytokines production (Chluba at al. Biomacromolecules. 2001, 2:800-805; Jesse et al., Adv. Materials, 2004, 16, 1507-1511). In this study, we checked first that PGA-α-MSH still also active when adsorbed on the top of the multilayered films (PGA-PLL). The objective here was to analyze the behaviors of the fibroblastic cells in term of adhesion and proliferation in contact of the multilayered film in the presence or absence of PGA-α-MSH. To further characterize PGA-α-MSH potential, its effect on cell adhesion was determined in our model. PGA-α-MSH multilayer films contribute to adhesion and proliferation of human pulp fibroblasts, whereas unmodified PGA or free α-MSH clearly still without effect on cell adhesion and proliferation as compared to control fibroblast cultures. This could suggest that PGA-α-MSH and α-MSH somehow interact differently with the cells. This was confirmed by the diffuse fluorescence observed after contact between FITC-PGA-α-MSH and pulp fibroblasts and, by the fact that cAMP levels were not significantly elevated after stimulation with PGA-α-MSH but were after treatment with MCR agonists, indicating that the effects of PGA-α-MSH are not specifically MCR receptor mediated. In fact, coupling α-MSH to PGA makes the C-terminal region of the peptide more accessible, enhancing the anti-inflammatory effects of the peptide without inducing cAMP accumulation as has already been suggested for the peptide alone (Getting at al. Exp. Ther. 2003, 306:631-635).

To understand more the cells behaviors when PGA-α-MSH is adsorbed on the top of the multilayered film (PGA-PLL)n, we have analyzed the effects of PGA-α-MSH incorporated into the multilayered film (PLL-PGA-ci-MSH)₁₀ as a reservoir for cells. We have also analyzed the effects, when PLL was replaced by Dendri Graft Poly-L-Lysines (DGLs) in the same architecture (DGL^{G4}-PGA-α-MSH)₁₀.

In FIG. 7, we analyzed proliferation and morphology of pulp fibroblasts growing on the surface of the multilayered films (PLL-PGA)₁₀ (A), (PLL-PGA-α-MSH)₁₀ (B), (DGL^{G4}-PGA)₁₀ (C) and (DGL^{G4}-PGA-α-MSH)₁₀ (D) followed by AlamarBlue and confocal microscopy (morphology). Our results indicated clearly an increase of cells proliferation in the presence of PGA-α-MSH when adsorbed on PLL or DGL^{G4} as a reservoir for cells. These results is on accordance of the results obtained by adsorption of only one layer of PGA-α-MSH on the top of the multilayered film (PLL-PGA)₅-PLL. To understand more these results, we analyzed at the nanoscale these multilayered films (Architectures, A, B, C and D).

First we analyzed by QCM dissipation versus frequency shift of the multilayered films during their build-up process. Successive adsorptions on top of a SiO₂-coated quartz crystal sensor were monitored in situ by QCM for (PLL-PGA)₁₀ (A), (PLL-PGA-α-MSH)₁₀ (B), (DGL^{G4}-PGA)₁₀ (C) and (DGL^{G4}-PGA-α-MSH)₁₀ (D) multilayered films. In FIG. 8, the Values of frequency shift and dissipation are collected and the linear regressions are added in the graph to follow the build.

From FIG. 8, when the evolution of dissipation is plotted versus evolution of frequency, it is evident that using DGL^{G4} or/and PGA-α-MSH during the build-up of the film have significantly modify the viscoelasticity value. The ratio R go form 1.0e⁻³ for (PLL-PGA)₁₀ (A) film to 10.0e⁻³ for (PLL-PGA-α-MSH)₁₀ (B) or (DGL^{G4}-PGA)₁₀ (C) films. Interestingly, by using both DGL^{G4} and PGA-α-MSH in the case of (DGL^{G4}-PGA-α-MSH)₁₀ (D) the ratio R increase until 14.0e⁻³ (Table 1).

The evolution of Δf/v showed a regular film deposition starting with the first layer of PLL. The increase in −Δf/v with the number of deposited layers suggested that regular film deposition occurred.

In the next step, atomic force microscopy (AFM) was used to get additional information about the structure of these multilayered architectures (data not shown).

Modification of dissipation can be correlated to increase the roughness of films analyzed by Atomic force microscopy (AFM) (Jessel et al. Proc. Nat. Acad. Sci. USA. 2006, 103:8618-8621). In this study, we have also analyzed the roughness and thickness of these nanostructured films (Architectures A, B, C and D) obtained by In situ AFM images height mode in buffer of these films (Table 1). The films made of alternating layers of PLL or DGL^{G4} and PGA or PGA-α-MSH were examined in situ by AFM in the liquid phase. The film thickness was evaluated by scratching the film with the AFM tip and estimated to 7.7 nm for (PLL-PGA)₁₀ without α-MSH 307 nm for (PLL-PGA-α-MSH)₁₀ and 532 nm for (DGL^{G4}-PGA-α-MSH)₁₀ in the presence of both DGL^{G4} and PGA-α-MSH deposited materials compared to the deposited DGL^{G4} in the architecture (DGL^{G4}-PGA)₁₀ without α-MSH estimated to only 8.2 nm. The roughness was also analyzed (Table 1) and still in line with the results obtained concerning the proliferation and the morphology of cells growing on the surface of these nanostructured films with an increase of the roughness (RMS) from 4.1 nm without α-MSH to 66.1 nm with α-MSH. Interestingly, by using DGL^{G4} the roughness (RMS) go from 2.3 nm without α-MSH to 18.7 nm with α-MSH. In this case, when we combine both DGL^{G4} and α-MSH, we do not have more roughness but more thickness. These results could be explained by an intra-layers process increasing the film cohesion and increasing the film hydration.

### Example 8

### Conclusion

Millions of teeth are saved each year by root canal therapy. Although current treatment modalities offer high levels of success for many conditions, an ideal form of therapy might consist of regenerative approaches in which diseased or necrotic pulp tissues are removed and replaced with healthy pulp tissue to revitalize teeth (Murray at al. J Endod. 2007 33:377-90).

Pulp fibroblast play a central role in signalling various aspects of tissue regeneration. The control of the pulp fibroblast response is fundamental to control the pulp inflammation. We have shown that free PGA-α-MSH can modulate the activation of human pulp fibroblasts and can regulate the inflammatory fibroblast's environment as well as the ability of the fibroblast to adhere.

We have also investigated whether PGA-α-MSH could induce adhesion when in contact with a multilayered film finding an increase in adhesion and proliferation whilst PGA alone or free α-MSH inhibited this mechanism.

In conclusion, this study highlights that PGA-α-MSH can not only modulate pro and anti-inflammatory cytokines but also promote adhesion of pulpal fibroblasts. Therefore these effects of PGA-α-MSH may have important regulatory functions in extracellular matrix composition.

Although the mechanism by which PGA-α-MSH can stimulate all of these responses in human pulp fibroblasts has not yet been elucidated, PGA-α-MSH may, nevertheless, have important regulatory functions to modulate pulpal inflammation which causes pulpitis followed by apical periodontitis.

We report here the first use of nanostructured and functionalized multilayered films containing α-MSH as a new active biomaterials for endodontic regeneration.

What is claimed is:

1. A method for endodontic regeneration and/or for treating a dental inflammatory disease, the method comprising administering an effective amount of a compound to an individual in need thereof, wherein said compound comprises an alpha-melanocyte stimulating hormone (α-MSH) peptide, coupled to a polypeptide consisting of a chain of about 15 to about 400 amino acids.

2. The method according to claim 1, wherein said amino acids are charged amino acids selected from the group consisting of aspartic acid, glutamic acid, lysine, arginine, histidine and ornithine.

3. The method according to claim 1, wherein said polypeptide is a polyanion or a polycation.

4. The method according to claim 3, wherein said polypeptide is a poly(glutamic acid) polypeptide (PGA).

5. The method according to claim 1, wherein said polypeptide is coupled to the N-terminal extremity of said α-MSH peptide.

6. The method according to claim 1, wherein said polypeptide is coupled to said α-MSH peptide through a maleimide coupling.

7. The method according to claim 1, wherein said α-MSH peptide comprises:
a) a sequence of SEQ ID NO: 1 or SEQ ID NO: 2; or
b) a sequence homologous to SEQ ID NO: 1 or SEQ ID NO: 2, wherein said homologous sequence is at least 60% identical to a sequence of SEQ ID NO: 1 or SEQ ID NO: 2; or
c) a fragment of at least 8 consecutive amino acids of the sequence of (a) or (b), wherein said fragment comprises amino acids 11 to 13 of SEQ ID NO: 1 or SEQ ID NO: 2; or
d) a fragment of at least 8 consecutive amino acids of the sequence of (a) or (h), wherein said fragment comprises amino acids 6 to 13 of SEQ ID NO: 1, SEQ ID NO: 2 or of the sequence homologous thereto.

8. The method according to claim 7, wherein said α-MSH peptide comprises a sequence of SEQ ID NO: 2.

9. (canceled)

10. The method according to claim 1, wherein said dental inflammatory disease is selected from the group consisting of pulpal diseases, pulpitis, dental caries, endodontic lesions, periradicular lesions, gingivitis, peridontitis, periimplantitis and ulceration.

11. The method according to claim 1, wherein said compound is for use in the treatment of a human individual.

12. The method according to claim 1, wherein said compound is for use in the treatment of a non-human individual.

13. The method according to claim 12, wherein said individual is a cat, and wherein said dental inflammatory disease is feline odontoclastic resorptive lesions (FORL).

14. A method for endodontic regeneration and/or for treating a dental inflammatory disease, the method comprising administering an effective amount of a pharmaceutical composition to an individual in need thereof, wherein said pharmaceutical composition comprises a compound and a pharmaceutically acceptable carrier, wherein said compound comprises an alpha-melanocyte stimulating hormone (α-MSH) peptide, coupled to a polypeptide consisting of a chain of about 15 to about 400 amino acids, and wherein said pharmaceutical composition is selected from the group consisting of a film, a varnish, a gel, a capsule, a solution and toothpaste.

15. The method according to claim 14, wherein said pharmaceutical composition is a nanostructured composition.

16. The method according to claim 14, wherein said pharmaceutical composition is a nanostructured composition and said nanostructured composition is a polyelectrolyte multilayered film.

17. The method according to claim 16, wherein said compound is embedded within the polyelectrolyte multilayered film.

18. The method according to claim 16, wherein said compound is adsorbed on the top of the polyelectrolyte multilayered film.

19. The method according to claim 16, wherein said polyelectrolyte multilayered film comprises at least one layer pair consisting of a layer of poly(lysine) polypeptides and a layer of poly(glutamic acid) polypeptides.

20. The method according to claim 16, wherein said polyelectrolyte multilayered film comprises at least one layer pair consisting of a layer of poly(lysine) polypeptides and a layer of said compounds.

21. The method according to claim 15, wherein said nanostructured composition comprises nanoparticles or capsules.

22. The method according to claim 14, wherein said method comprises the step of topically applying said pharmaceutical composition to said individual.

23. The method according to claim 14, wherein said method comprises the step of administering by injection said pharmaceutical composition to said individual.

24. The method according to claim 16, wherein said compound is embedded within the polyelectrolyte multilayered film at each layer pair of said film.

25. The method according to claim 22, wherein said method comprises before the step of applying topically said pharmaceutical composition to said individual, the step of chirurgically cleaning a diseased tooth or lesion in said individual.

26. The method according to claim 23, wherein said method comprises before the step of administering by injection said pharmaceutical composition to said individual, the step of chirurgically cleaning a diseased tooth or lesion in said individual.
